(19) United States
(12) Patent Application Publication
(10) Pub. No.: US 2025/0191774 A1
(43) Pub. Date: Jun. 12, 2025

(54) METHODS AND SYSTEMS FOR DETECTING PRESCRIPTION ANOMALIES IN REAL TIME

(71) Applicant: DRFIRST.COM, INC., Rockville, MD (US)

(72) Inventors: Ying Zhou, Cary, NC (US); Anthony Gordon Brooke, Redding, CT (US); Fei Li, Brooklyn, NY (US); Dat Q Duong, Germantown, MD (US); Daniel Douglas Garmat, Silver Spring, MD (US)

(21) Appl. No.: 18/972,457

(22) Filed: Dec. 6, 2024

Related U.S. Application Data
(60) Provisional application No. 63/607,920, filed on Dec. 8, 2023.

(51) Int. Cl. G16H 50/30; G16H 20/10; G16H 80/00

(52) U.S. Cl. CPC G16H 50/30; G16H 20/10; G16H 80/00

(57) ABSTRACT

A system and method for detecting prescription fraud in real time utilizes machine-learned models to analyze prescription histories and identify outlier behavior among clinicians. The system generates outlier scores representing the probability that a clinician's prescribing behavior deviates from norms. These scores can be used to evaluate prescription events, generating event scores that indicate the likelihood of illicit activity. The system employs a graph database to model relationships between clinicians, pharmacies, medications, and prescription events. Upon determining that an outlier or event score meets an alert threshold, the system can deny access to electronic prescription applications or alert pharmacies to potential fraud. Reinforcement learning is used to improve model accuracy, minimizing false positives while identifying complex fraud patterns.

### RELATED APPLICATION

This application is a non-provisional of, and claims priority to, U.S. Provisional Application No. 63/607,920, titled “Methods and Systems for Detecting Prescription Fraud in Real Time,” filed Dec. 8, 2023, the disclosure of which is incorporated herein in its entirety.

### TECHNICAL FIELD

The present teaching relates to methods, systems, and programming for identifying anomalies in prescription activity. Particularly, the present teaching is directed to building and using machine-learned models to detect and monitor clinician outlier behavior and to provide a probability that a prescription event is a result of illicit activity as a prescription is generated.

### BACKGROUND

Prescription drug abuse has many negative effects beyond the illegality of any particular prescription event, a prescription event being a particular medication prescribed for a particular patient by a particular clinician, but such abuse is difficult to combat. Pharmacies filling prescriptions may have little to no insight into whether any single transaction is legitimate or not and, therefore, have no basis for questioning a transaction. Similarly, clinicians may have no insight into theft of their professional identity.

### SUMMARY

Implementations provide a real-time analysis of prescription providers (clinicians) and prescription events using machine-learned models. The models can analyze prescription histories and recognize patterns humans are unable to identify. One model can provide respective outlier scores for clinicians, the outlier score representing a degree to which the unsupervised learning model predicts the clinician is a risk for illicit prescription events based on an analysis of the clinician's prescribing behavior (done by the model) that falls outside of norms for similar clinicians. The outlier score represents an actionable prediction, which can be used to initiate an investigation, to alert a clinician of potential professional identity theft, to evaluate a particular prescription event, suspend e-prescription activity, etc. As used herein a clinician refers to a physician, a nurse practitioner, a dentist, or any other medical provider authorized to prescribe medications. Implementations may also provide an event score for a particular prescription event, the event score being an actionable prediction of the risk of the prescription event being a result of illicit activity. One or both models may use a vectorized representation of entities involved in a prescription event to analyze prescription histories/events. Put another way, the models may use a clinician vector, a pharmacy vector and a prescription event vector, to analyze prescription events for a clinician, where each vector represents attributes about that entity. Some implementations may use a medication vector. Some implementations may use a patient vector. Some implementations may use a sponsor vector. Some implementations may use a graph database to represent the clinicians, pharmacies, medications, and prescription events. The outlier score of a clinician may be calculated periodically and can be stored as an attribute of the clinician. The outlier score can be one attribute considered by the model that analyzes prescription events. Both models may use reinforcement learning to improve the predicted scores. This combination enables the system to minimize flagging legitimate prescriptions as suspicious while identifying new patterns in prescriptions as suspect that on their face appear legitimate.

A technical problem with identifying suspicious prescription events is timeliness. Identifying a prescription as suspicious after the patient has already picked up the prescription is ineffective, as it does not prevent fraudulent transactions, or in other words, transaction anomalies. Another technical problem with identifying suspicious prescription events is the patterns indicating anomalies (fraud) can be complex, involving interconnected relationships that, individually may not raise suspicions, but collectively indicate a suspicious pattern. These interconnected relationships may also change frequently, as bad actors adjust to rules based on only a few dimensions. Implementations provide a technical solution to these problems by providing machine-learning models that evaluate clinicians for outlier behavior, a factor used in real-time evaluation of a particular prescription event so the legitimacy of the prescription can be questioned before it is filled. In other words, implementations can determine the outlier behavior for a specific prescription in at most a few minutes, and in some implementations even less than a minute from when a pharmacist receives the prescription for fulfillment. The machine-learning models can identify complex patterns that can include hundreds of dimensions, something humans are not capable of doing.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 describes a high-level depiction of a system configuration, according to a disclosed embodiment;

FIG. 2 illustrates a flowchart of an example process, according to a disclosed embodiment; and

FIG. 3 illustrates a flowchart of an example process, according to a disclosed embodiment.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, systems, components, and/or circuitry have been described at a relatively high-level, without detail, in order to avoid unnecessarily obscuring aspects of the present teachings.

To solve the problems associated with identifying prescription events as illicit, such as identifying complicated patterns of activity indicative of fraud, identifying and alerting cases of likely professional identity theft, accurately identifying a prescription event as suspect, and providing an opportunity to interrupt a suspected illicit prescription event, implementations utilize a graph database that captures the attributes of prescribing clinicians, attributes of pharmacies that fill prescriptions, attributes of medications, and attributes of prescription events as well as the relationships between these different entities. Some implementations include training and using machine-learned models to cluster clinicians and use the clusters and the graph database to identify clinicians with an outlier prescribing history. Put another way, implementations may cluster clinicians into similarity clusters based on attributes of the clinician. The model may then analyze the prescription history of a clinician and compare that history with the prescription histories of other clinicians in the cluster to determine an outlier score for the clinician. The outlier score may represent a probability or prediction or extent to which a clinician prescribes within normal boundaries for the cluster, or in other words the extent to which a clinician's prescribing history represent an outlier prescribing history. In some implementations, the outlier score may be calculated on a periodic basis. In some implementations, the outlier score can be calculated by practice for a clinician, by an e-prescribing application used for a clinician, or by a combination of practice/e-prescribing application. Thus, for example, when a clinician is associated with two different e-prescribing applications, the clinician may be associated with two outlier scores, one for each e-prescribing application. Similarly, a clinician may have two or more outlier scores where the clinician is associated with two or more practices. In some implementations, the outlier score can be associated with a combination of practice and e-prescribing application. The outlier score may be an attribute stored for a clinician, i.e., an outlier score attribute. In some implementations, the outlier score may be an attribute of a clinician node in a graph database. In some implementations, a clinician may be notified if their outlier score drops by a predetermined amount. In some implementations, a clinician may be referred to an investigator if the outlier score meets an alert threshold. In some implementations, a clinician may be notified if a new outlier score is added for a new e-prescribing application or a new practice. Such notifications may alert the clinician to professional identity theft.

The outlier score can be used to boost or lower the suspiciousness of a particular prescribing event. Put another way, clinicians with low outlier scores (within prescribing norms) may lower the event score for a particular event while a high outlier score (outside prescribing norms) may boost the event score for a particular event. For example, a particular prescription event may have an event score that is borderline suspicious, but if the prescribing clinician has a low outlier score, the event score may be lowered so it would not meet an alert threshold but a high outlier score for the prescribing clinician may boost the event score so it would be an alert threshold. The model that generates an event score may thus take into account the outlier score (or scores) for a clinician when generating the event score.

Implementations also include a machine-learning model that generates event scores for prescription events. The event score represents a probability that the prescription event represents illicit activity, or in other words represents a suspicious event. The machine-learning model may operate on a graph database. The graph database includes nodes (entities) connected by edges representing relationships between the nodes. The graph nodes may represent one of clinicians (as clinician nodes), pharmacies (as pharmacy nodes), and medications (as medication nodes). The nodes may be connected by edges representing prescription events. In such an implementation, each edge may have an attribute that uniquely identifies the prescription event. Thus, a clinician may be connected to a pharmacy and a medication by edges having the same event identifier. In some implementations, some of the graph nodes may also represent prescription events. In such an implementation, the event node may have a unique identifier and edges may connect the clinician, the pharmacy, the medication, and the event node. Whether an event is represented as an edge or a node, the prescription event can also have attributes, such as a date/time prescribed, a location, an e-prescribing application used (e.g., an identifier of an electronic prescription, or e-prescription, application), a practice identifier associated with the application used, a patient identifier and attributes (unless the graph also includes patient nodes, in which case an edge may connect the patient node, which includes patient attributes, with the event node or the edge may represent an event connecting the patient node with the clinician, pharmacy, and medication nodes related to the event), a sponsor identifier and attributes (unless the graph also includes sponsor nodes, in which case an edge may connect the sponsor node, which includes sponsor attributes, with the event node), and/or other data fields related to the prescription event not represented by nodes in the graph. etc.

A clinician node may also include attributes, such as a name for the clinician, an NPI number, an outlier score, a cluster assignment (e.g., identifying a specialty for the clinician), a location etc. A pharmacy node may include attributes, such as the location of the pharmacy. A medication node may include attributes, such as dose, format, etc. (so that different dosages/formats of a medication are represented by different nodes). In some implementations, the attributes may be represented in vector form.

In some implementations, a machine learning model is trained to identify patterns of illicit activity, for both clinicians and for prescription events, based on the graph database. Such models can be used to evaluate new prescription events, e.g., as the prescription is sent to the pharmacy or in response to an express request from the pharmacy, and assign the new prescription event an event score.

To train the event model, reinforcement learning may be used. For example, medication nodes that represent drugs with high street value, and are therefore often the target of illicit prescription activity, may be identified to the model, as are clinicians and pharmacies known (e.g., via law enforcement prosecutions) to be involved in illegal prescription activity. The model may use reinforcement learning to analyze the graph and learn patterns that result in (describe) highly suspicious prescription events, or in other words prescription events likely to represent illicit activity. Such events may be given an event score that represents high risk of illicit activity, e.g., a high probability of being a suspicious prescription event. In some implementations, the event score is a probability, e.g., ranging between zero and 100, where 100 is a highest probability of the prescription event representing illicit activity.

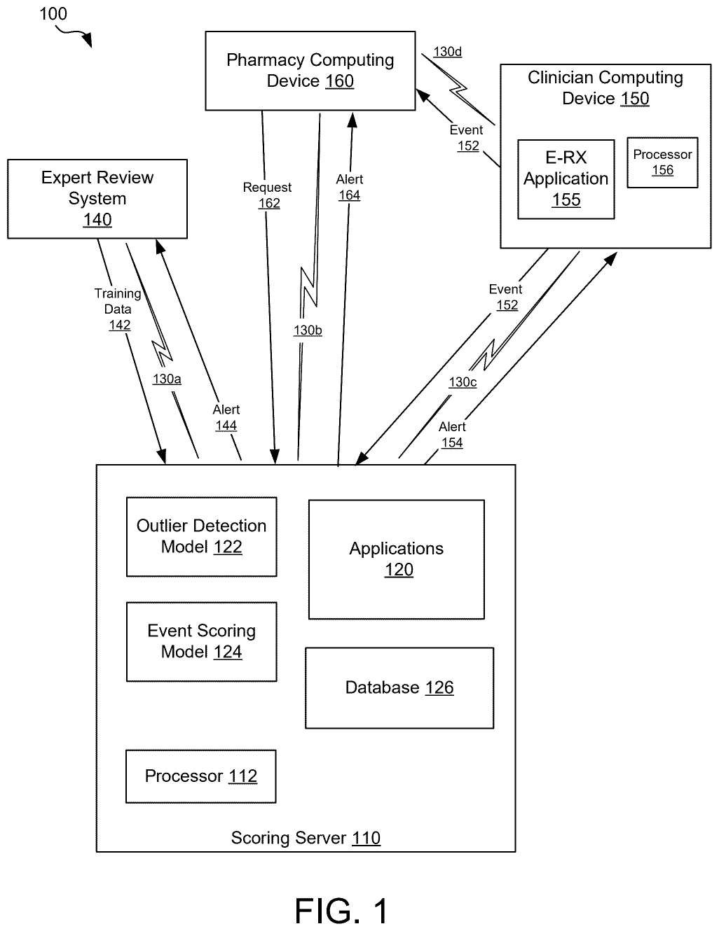

As shown in FIG. 1, a networked environment 100 may include a number of computing devices that are in data communication with each other through one or more communications channels 130, e.g., 130a through 130d. The communication channels 130 can represent connections via a network, the Internet, a local area network (LAN), a wide area network (WAN)) WI-FI, a mobile network, an intranet, etc., or some combination of these. For example, communication channel 130a may represent connections using a LAN, a mobile network, an intranet, the Internet, etc. allowing an expert review system 140 to communicate with the scoring server 110. Communication channel 130b, which allows the scoring server 110 to communicate with one or more pharmacy computer systems 160, may likewise represent connections using one of or any combination of different communication channels.

Communication channel 130c, which allows the scoring server 110 to communicate with one or more clinician computing devices 150, and communication channel 130d, which allows a clinician computing device 150 to communicate with a pharmacy computer system 160, may represent connections using one of or any combination of different communication channels.

The scoring server 110 may include a computing device, such as a computer, a server or a number of communicatively connected distributed servers, a mainframe, etc., that has one or more processors 112 (e.g., a processor formed in a substrate) configured to execute instructions stored in memory, such as main memory, RAM, or disk. The instructions may be stored as modules, engines, or applications and may provide functionality modules or engines and may provide functionality typical of an enterprise system, including an EMR.

The scoring server 110 may include one or more applications 120, that respond to requests 162 from the pharmacy computer system 160, receive data, such as prescription event 152 and training data 142, update and maintain databases, such as the database 126, manage training and fine-tuning of the outlier detection model 122 and/or the event scoring model 124, and use the outlier detection model 122 and/or the event scoring model 124 in inference mode to generate scores, use the scores to update databases and/or to provide alerts, as described herein.

The clinician computing device 150 may be a personal computing system, a terminal, a laptop, a tablet, a mobile device such as a smartphone, or a wearable computing device (e.g., a smart watch or smart glasses). The clinician computing device 150 may include one or more processors 156 (e.g., a processor formed in a substrate) configured to execute instructions stored in memory such as main memory, RAM, flash, cache, or disk. The clinician computing device 150 may also include input devices, such as a microphone, a keyboard (virtual or physical), a touch screen, a mouse, a camera, a voice recorder, etc. The clinician computing device 150 also includes a display or other output device, such as a speaker, lights, or haptic feedback device. The clinician computing device 150 may also include one or more applications that perform various functions, e.g., a browser, a word processing program, a spreadsheet program, an email client, a mobile application etc. The applications can include electronic prescription (E-RX or e-prescription) application 155. The E-RX application 155 may be configured to allow a clinician to enter a prescription for a patient and send the prescription directly to a pharmacy computer system 160, e.g., as prescription event 152. In addition, the E-RX application 155 may be configured to send information and metadata for the prescription event 152 to the scoring server 110. In some implementations, the information and metadata sent to the scoring server 110 may include information or metadata not included in the prescription event 152 sent to the pharmacy computer system 160.

The scoring server 110 may use the prescription event 152 to update one or more databases, including the database 126. In some implementations, the updates may be adding edges representing the prescription event between nodes representing entities identified in the prescription event 152, such as a clinician node, a pharmacy node, and one or more medication nodes. In some implementations, a patient node may be identified by the prescription event 152. In some implementations, the prescription event 152 may include attributes of the patient. In some implementations, one or more sponsor nodes may be identified by the prescription event 152. A sponsor entity represents a person or organization responsible for at least partial payment of the medication. In some implementations, the prescription event 152 may include attributes for one or more sponsors. In some implementations, the updates may be adding a node representing the prescription and linking that added node to nodes representing entities identified in the prescription event 152. In some implementations, the updates may be adding records to a data structure that represents a prescription event. Such records may identify the clinician, pharmacy, and/or medication related to the prescription event. In some implementations, the scoring server 110 may receive data from another source (not illustrated) that provides information for adding new clinicians, new pharmacies, and/or new medications.

The scoring server 110 may include an outlier detection model 122. The outlier detection model 122 may be a machine learning model configured to analyze attributes of a clinician (e.g., a vector representation of the clinician) and attributes describing prescription events for the clinician (e.g., a vector representation of the prescription events). In some implementations, the outlier detection model 122 may be configured to also analyze attributes of the patient (e.g., a vector representation of the patient) in addition to the clinician vector and the event vector. In some implementations, the outlier detection model 122 may be configured to also analyze attributes of one or more sponsors (e.g., a vector representation of the sponsor) in addition to the clinician vector and the event vector. Put another way, the outlier detection model 122 may be configured to use the clinician vector and the event vector or to use the clinician vector, the event vector and one or more of a patient vector or a sponsor vector as input. In some implementations, the outlier detection model 122 may be configured to perform empirical-cumulative distribution-based outlier detection (ECOD). The outlier detection model 122 may be an unsupervised model trained using one-shot or few-shot learning and/or reinforcement learning. In some implementations, the outlier detection model 122 may include or may be based on clustering clinicians, in other words grouping similar clinicians together based on the attributes of the clinicians, i.e., based on the vector representation of the clinicians. The clustering can be done as part of the outlier detection model 122 or may be done before running the outlier detection model 122. In some implementations, clustering may be used to initially identify outliers, which can be used in one-shot or few-shot learning for an ECOD model.

This vector representation of a clinician may include dimensions representing one or more aspects of the clinician. Example aspects include a specialty (or specialties) the clinician practices in, a location of the clinician, an enterprise medical system (EMS) associated with the clinician, a last e-prescription event date (e.g., the date of the most recent prescription event associated with the clinician, which represents how active the clinician is), a first e-prescription event date (e.g., data representing how long the clinician has been prescribing), average number of prescriptions over some time period (e.g., a month (30 days), 90 days, six months, a year) since the first e-prescription event date (e.g., how often does this clinician prescribe medications), the actual number of prescriptions for the clinician over a most recent time period (e.g., a month, 90 days, six months, etc.), the unique medications prescribed during a most recent time period (e.g., a Family Practice clinician who prescribes only two different medications during the last 90 days, one of which is a controlled substance, may have a higher outlier score than another Family Practice clinician who prescribes a dozen different medications), and/or other such information. To generate an outlier score, the outlier detection model 122 may analyze a prescription history for the clinician. In implementations that generate clusters of clinicians, the outlier detection model 122 may compare a clinician's prescription history against other clinicians in the cluster. In implementations that use distribution-based outlier detection (e.g., ECOD), the weights of the model may be used to evaluate the clinician's prescription history. A prescription history represents the electronic prescriptions signed by the clinician. As used herein, a clinician may be understood to represent an account with an enterprise medical system. An individual clinician (i.e., uniquely identified by a national provider identifier, or NPI) may have multiple accounts. For such a clinician, the clinician attributes (the clinician vector) may include data fields specific to that account and the outlier detection model 122 may analyze prescription history events tied to the account. Thus, the outlier detection model 122 may generate two different outlier scores, one for each account, for the same NPI. In some implementations, the outlier detection model 122 may consider all prescription events for a single provider, e.g., for a unique NPI.

The prescription history may be a recent history, e.g., prescriptions written within a most recent time period (e.g., 2 months (or 60 days), 3 months (or 90 days), 6 months, etc.). In some implementations, a clinician may need a minimum number of prescriptions within the time period to be considered; if a clinician does not have a recent prescribing history (e.g., insufficient prescription events during the time period) then the outlier detection model 122 may not generate an outlier score for that clinician, as there is insufficient data on which to base the score. In implementations that rely on clustering, that clinician's prescription events may be excluded from the determination of an average prescription history for the cluster. The prescription history events may also be represented as a vector or as vectors. The attributes of the prescription history events represented in the vector(s) can include a date of the prescription, the medication prescribed, a dosage, a pharmacy identifier, and/or other attributes. In some implementations, the attributes of the event may also include attributes representing the patient for whom the prescription was written. Thus, for example, in some implementations, an age of the patient may be represented, a location of the patient, and/or other demographics of the patient. In some implementations, the attributes of the patient may be represented in a different vector, such as the event vector. In some implementations, the attributes of the event may also include attributes representing one or more sponsors responsible for payment of the prescription. Put another way, a sponsor can be a payer such as an insurance company, a government agency, the patient, another person, etc. Thus, for example, in some implementations, a location of the sponsor may be represented, a type of the sponsor, and/or other attributes of the sponsor. In some implementations, attributes of the sponsor may be represented in a different vector, such as an event vector. The outlier detection model 122 also considers attributes of the pharmacy. The pharmacy identified by the prescription may thus have its own vector, describing attributes of the pharmacy. The attributes included in the pharmacy vector can include a physical location (address) of the pharmacy, a type of the pharmacy (e.g., mail order, clinician office, supermarket, drug store, etc.), and/or other attributes of the pharmacy.

In some implementations, the outlier detection model 122 may compare a clinician's prescribing history within that time period against an average prescription history for the cluster. In other words, the outlier detection model 122 may determine to what extent the clinician's prescribing history, represented in the recent time period, represents an outlier when compared with the prescribing histories of the cluster. In some implementations, the outlier detection model 122 uses the vector representations of the clinician, the prescription events (including patient, sponsor, and/or pharmacy attributes, either as part of the event vector or as separate vectors) to provide the outlier score. Such implementations may use distribution-based outlier detection methods. In a distribution-based outlier detection method, clinician's activity may still be compared with other clinicians having the same specialty.

The extent to which the clinician's prescribing history represents an outlier is expressed as an outlier score. Thus, the outlier detection model 122 is configured to provide, as output, an outlier score for the clinician. In some implementations, the outlier score may be associated with a confidence score indicating the strength/reliability of the outlier score. In some implementations, the outlier detection model 122 may be configured to generate a respective outlier score for each account associated with a clinician. For example, a clinician may have an account with an enterprise medical records system (EMS), which may use one or more e-prescription applications (e.g., E-RX application 155). Each account may be represented by a clinician vector (e.g., with different last prescription dates, different first prescription dates, etc.), different location, etc. The outlier detection model 122 may be configured to generate an outlier score for each account represented in the prescription history for the clinician. Because bad actors can steal the NPI of a clinician and open new accounts using a different enterprise medical system (and potentially different e-prescription applications), or because a clinician could use one account for legitimate prescriptions and another account for illicit activity, these separate outlier scores, e.g., by practice, account, or combinations of practice/accounts, may be used to identify and target professional identity theft or separate illegal activity. In some implementations, the scoring server 110 may obtain outlier scores for clinicians using the outlier detection model 122 periodically, e.g., weekly, bi-weekly, etc.

The scoring server 110 may store the outlier score as an attribute of a clinician. The outlier score may be an attribute related to a clinician node in an implementation where the database 126 is a graph database. In some implementations, the outlier score may be an attribute related to a clinician in the database 126. In some implementations, the outlier score may be displayed to a clinician, e.g., as part of E-RX application 155 or an EMS. The scoring server 110 may perform an action in response to determining that the outlier score meets a threshold. In some implementations, the threshold is an alert threshold that represents a likely outlier and if the outlier score meets the alert threshold, an alert may be provided to an expert for review, e.g., a human investigator and/or an artificial intelligence expert. Alert 144 is an example of an alert to an expert. The expert review system 140 may be a system that allows experts to review a prescription event and/or a clinician's prescribing history. The experts can include a team associated with the scoring server 110. The experts can include an AI model associated with the scoring server 110 or accessible to the scoring server 110 that is specially trained to review a prescription event. The experts may not be associated with the scoring server. In one implementation, a team associated with the scoring server 110 may review the clinician's prescribing history (a clinician identified in the alert 144) and determine whether the prescribing history and/or the clinician warrants further investigation. An expert may also review the clinician's prescribing history and provide information used to fine-tune the outlier detection model 122 (e.g., labeling (positively identifying) the clinician as an outlier, as not an outlier, etc.) and/or used for reinforcement learning of the outlier detection model 122. The alert 144 may be in the format of a report or list.

In some implementations, the scoring server 110 may include an application 120 that selects clinicians for review. For example, the scoring server 110 may select clinicians with outlier scores for expert review. The expert may be a human. The expert may be another intelligent model (e.g., another A/I model). In some implementations, the expert review system 140 may include a user interface configured to work with the scoring server 110 to facilitate the review. In some implementations, the scoring server 110 may select clinicians with outlier scores randomly. In some implementations, the scoring server 110 may select clinicians with outlier scores that have a low confidence. In some implementations, the scoring server 110 may select clinicians with borderline outlier scores for review by experts. An outlier score may be a borderline outlier score when the outlier score meets a trustworthiness threshold but is within a predetermined range of the trustworthiness threshold. In other words, the scoring server 110 may select outlier scores that are close to not meeting the trustworthiness threshold. In some implementations, the scoring server 110 may use a combination of the preceding factors to select clinicians for review by experts. In some implementations, a hundred, two hundred, etc. may be selected per week. A result of the expert review may be a label for the clinician (e.g., a positive identification of the prescription history as an outlier/not an outlier) used to further train the outlier detection model 122.

In some implementations, if the outlier score meets the alert threshold the scoring server 110 may revoke or suspend the ability of the clinician to continue using the E-RX application 155. In some implementations, a higher threshold (e.g., a suspension threshold) may be used before suspending use of the E-RX application 155 or the outlier score may need a higher confidence score to suspend the use of the E-RX application 155.

In some implementations, the threshold may be a change threshold, e.g., representing a difference between a previous outlier score for the clinician and a current outlier score for the clinician. In such implementations, if the difference between the outlier scores meets the change threshold, an alert may be provided to the clinician e.g., alert 152. In implementations that generate a respective outlier score for different accounts, an alert may be sent to the clinician when a new outlier score is generated for a new account, i.e., the outlier score is generated for the first time for an account. The alert can be a confirmation request, e.g., asking the clinician to reply if the account associated with the new outlier score is not a result of the clinician's activity. If a reply is received, this can be used for reinforcement learning or fine tuning of the outlier detection model 122.

One or more of the pharmacy computer systems 160 or another computing system may send a request 162 to the scoring server 110 before filling a prescription. In some implementations, the request 162 is implicit, e.g., is implicitly generated by receipt of a new prescription event 152. The requests 162 may include or identify a prescribing event, such as prescription event 152. The scoring server 110 may use the event scoring model 124 to determine an event score for the prescription event identified/sent as part of the request 162. The event scoring model 124 may be a machine-learning model that uses the database 126 to identify patterns that likely represent illicit prescriptions. In some implementations the patterns may be patterns in a graph. The event scoring model 124 may be provided with a prescription event as input and provide an event score for the prescription event as output. The event score can be associated with a confidence score. The prescription event can include at least identifiers for the clinician, the pharmacy, and the medication(s) associated with the event. The prescription event may include attributes about the patient (e.g., age, gender, location, etc.). The prescription event may include an identifier for the patient, e.g., where the database 126 includes records or nodes representing patients. The prescription event may include attributes about the sponsor(s) (e.g., a sponsor type, a location, a sponsor priority (primary, secondary, tertiary), etc.). The prescription event may include an identifier for one or more sponsors, e.g., where the database 126 includes records or nodes representing sponsors.

The event scoring model 124 may be trained using known examples of illicit prescriptions (e.g., cases where law enforcement has identified and prosecuted clinicians or pharmacies, represented by training data 142) and may be provided with feedback (e.g., from results of investigations by an expert review system 140) on whether a particular event score was accurate. In some implementations, a prescription event may receive an event score with a low confidence score. When confidence is low, the scoring server 110 may report the prescription to an evaluator (a human expert, another specially trained A/I model) which can provide feedback to the model (e.g., provide a ground truth label for the prescription event) for fine-tuning the event scoring model 124. In some implementations, the scoring server 110 may include an application 120 that selects prescription events for review. For example, the scoring server 110 may select prescription events with event scores for expert review. In some implementations, the expert review system 140 may include a user interface configured to work with the scoring server 110 to facilitate the review. In some implementations, the scoring server 110 may select prescription events randomly for review. In some implementations, the scoring server 110 may select prescription events with event scores that have a low confidence for review. In some implementations, the scoring server 110 may select prescription events with borderline event scores for review by experts. An event score may be a borderline event score when the event score fails to meet an alert threshold but is within a predetermined range of the alert threshold. In other words, the scoring server 110 may select event scores that are close to meeting the alert threshold. In some implementations, the scoring server 110 may use a combination of the preceding factors to select prescription events for review by experts. In some implementations, a hundred, two hundred, etc. may be selected per week. A result of the expert review may be a label for the prescription event. The label and the prescription event may be used to further train the event scoring model 124.

Other forms of feedback may also be used, such as a clinician responding to an alert 154 about a potentially suspicious prescription. If the clinician replies that the prescription was authorized, and the clinician has an outlier score that does not meet an alert threshold (i.e., the clinician has an outlier score indicating the clinician is considered trustworthy), the prescription event may be labeled as not-suspect (e.g., an event score of 0 or some adjustment to the event score based on the clinician's outlier score), but if the clinician indicates the prescription was not authorized, the prescription event may be labeled as suspect (e.g., an event score of 100 or some adjustment to a score of 100 based on the clinician's outlier score). The event scoring model 124 may also use reinforcement learning to identify new patterns of illicit prescription activity.

The scoring server 110 may respond to the requests 162 with an alert 164. The alert 164 may include an event score for the prescription event. The alert 164 may include a message generated for the event score. The message may be based on whether the event score meets an event threshold. When the event score does meet the event threshold, the message may be an indication that the pharmacist may want to initiate further investigation before completing the prescription. In some implementations, when the event score meets the event threshold the message may block the completion of (block the pharmacist from providing) the prescription. In some implementations, an alert is only sent when the event score meets the event threshold.

The environment 100 represents an example environment. Although illustrated with specific components in FIG. 1, the environment 100 may include additional components not illustrated, or may not include all elements shown. In addition, some components may be combined into a single component. For example, one or more of the pharmacy computer systems 160 may be combined with the scoring server 110. Moreover, the scoring server 110 is understood to be configured to comply with any applicable laws, regulations, or other conditions relating to the data input and the data obtained from the clinician computing device 150, pharmacy computer system 160, and expert review system 140.

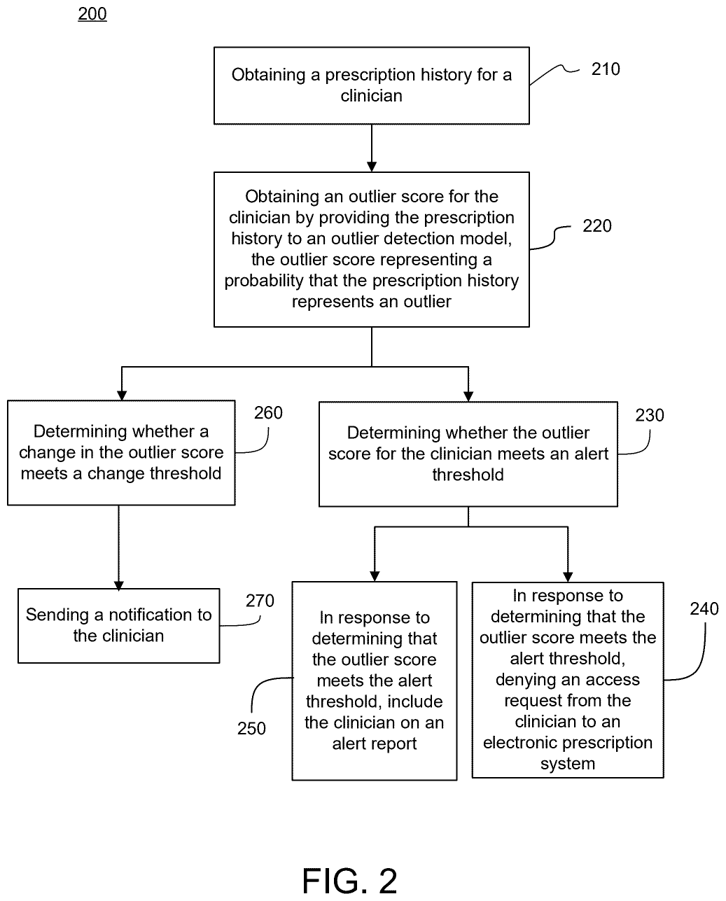

FIG. 2 illustrates a flowchart of an example method 200 of generating an outlier score for a clinician, according to a disclosed embodiment. Method 200 may be performed by a scoring server, such as scoring server 110 of FIG. 1. Method 200 may be performed periodically, e.g., weekly, bi-weekly, monthly, etc. Method 200 evaluates a clinician's prescribing history against other clinicians in the cluster to determine whether the clinician represents an outlier for the cluster. For example, at step 210, a prescription history for a clinician is obtained. The prescription history may represent a relatively recent period of time, e.g., two months, three months, six months, etc. Using less than an entire history may enable the system to detect a shift in the outlier score. In some implementations, more weight may be given to relatively recent prescription events. In some implementations, a decay may be applied to prescription events in the clinician's prescribing history based on the age of the prescription event, causing the oldest events to have little or no weight. The prescription history may be associated with an account.

At step 220, the system may provide the prescription history to an outlier detection model. The clinician and the prescription history can be vectorized and provided to the model. The model may generate an outlier score based on the attributes of the clinician, e.g., comparing the prescription history of the clinician with prescription histories of similar clinicians. When a clinician is an outlier, this is an indication that the clinician's pattern of prescribing may be suspicious because it lies outside the norm for the cluster. A clinician may be considered an outlier when the prescription history is more than a predetermined number of standard deviations from the norm for similar clinicians. An outlier score can be an attribute (dimension) of a clinician considered in evaluating prescription events by the clinician to determine an event score. As part of step 220, the system may store the outlier score as an attribute of the clinician. In some implementations, the system may store a history of outlier scores. The outlier score can be used to refer clinicians for investigation. For example, at step 230, the system may determine whether the outlier score meets an alert threshold. If the outlier score meets the alert threshold, at step 250 the clinician may be included on an alert list (report) so that an expert can review the clinician's prescription history, as described herein. In some implementations, at step 240, if the outlier score meets the alert threshold the clinician may be prevented from using an electronic prescription application. In other words, the system may deny an access request from the clinician to the electronic prescription application. In some implementations, the alert threshold for suspension may be a threshold (a suspension threshold) that represents a higher confidence in the outlier score and/or an outlier score that represents higher deviance from the norm. In other words, before suspending use of an e-prescribing application (denying an access request), the outlier score may need to represent high probability/confidence that the clinician's prescription history includes illicit activity.

The outlier score can be used to push potential fraud notifications to a clinician. For example, bad actors can steal a clinician's professional identity, e.g., by signing up as the clinician under a practice and/or e-prescribing application not used by the clinician. The bad actor may then begin writing illicit prescriptions using the different e-prescribing application without the clinician's knowledge. But because an NPI number is needed for e-prescriptions, and because an NPI is unique to a clinician, the scoring server 110 can trace the illicit prescription events back to the clinician, and these events will impact the clinician's outlier score, i.e., changing the score to reflect outlier behavior. Thus, at step 260, the system may determine whether a change in the outlier score (toward outlier behavior, i.e., not meeting a trustworthiness threshold) over some predetermined period of time meets a change threshold. If the change in the outlier score meets the change threshold, at step 270, the system can trigger an alert to the clinician.

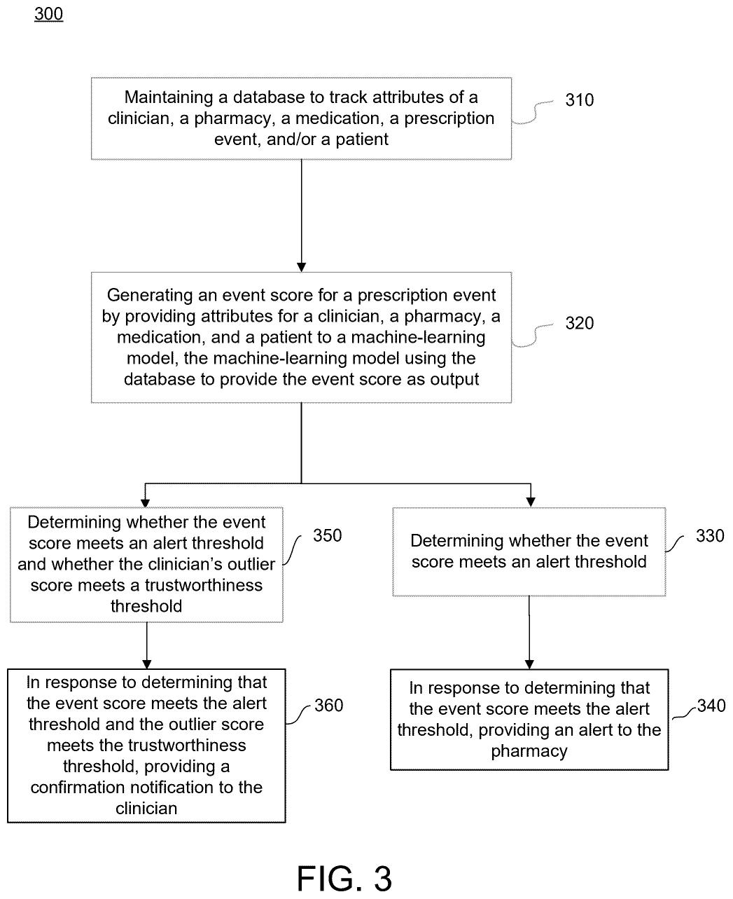

FIG. 3 illustrates a flowchart of an example method 300 of generating an event score for a prescription event, according to a disclosed embodiment. Method 300 may be performed by a scoring server, such as scoring server 110 of FIG. 1. At step 310, method 300 includes maintaining a graph database, such as database 126 of FIG. 1. The database may store attributes for clinicians, attributes for pharmacies, attributes for medications, and attributes for prescription events. One of the attributes for a clinician may be the outlier score for the clinician. A prescription event may link a clinician to a pharmacy and one or more medications. In some implementations, the database stores attributes about patients. In some implementations, patient attributes may be included in prescription events. In some implementations, the database stores attributes about sponsors. In some implementations, sponsor attributes may be included in prescription events. The system may generate vectors from the attributes in the database. Each vector may describe attributes of an entity involved in the prescription event, e.g., a clinician and a pharmacy. A medication entity may also be included. A patient entity may also be represented. A sponsor entity may also be represented. In some implementations, the database may be a graph database where each node represents a clinician, a pharmacy, or a medication. Some implementations may include other types of nodes, such as patient nodes and/or sponsor nodes. In some implementations, the edges connecting nodes represent prescription events. Maintaining the graph database includes adding new prescription events to the graph database. Each node may have attributes describing the node. In some implementations, the nodes are stored as vectors, e.g., with the attributes for the node reflected in the vector.

At step 320, method 300 may include using an event scoring model to provide an event score for a new prescription event. For example, before adding the prescription event to the database, or as part of adding the prescription event to the database, a machine-learning model may be used to determine an event score for the prescription event. In some implementations, the system may obtain attributes describing the pharmacy, the clinician, the medication, and the prescription event and generate vectors from these attributes to provide to the model. In some implementations, the machine-learning model performs analysis of the graph database. In such an implementation, identifiers for the nodes representing the pharmacy, the clinician, and the medications may be provided to the event scoring model, along with attributes of the prescription event. Attributes of the prescription event can include, for example, the date, attributes of the patient or a patient identifier if patients are represented as nodes in the graph. In some implementations, attributes of the prescription event can include attributes of one or more sponsors or a sponsor identifier if sponsors are represented as nodes in the graph. The event scoring model may provide, as output, an event score for the prescription event. The event score may be provided with a confidence score. The event scoring model may calculate the event score based on similarities between relationships and attributes of the prescription event and relationships and attributes that represent illicit activity. Thus, the event score represents a probability that the prescription event represents illicit activity.

If the event score meets an alert threshold, the pharmacy filling the event may be notified, as described herein. For example, at step 330, the system may compare the event score to an alert threshold. If the event score meets the alert threshold, at step 340, the system may provide an alert to the pharmacy. In some implementations, the event score and an outlier score can be used to push potential fraud notifications to a clinician. For example, in some implementations, at step 350 the event score may be compared against an alert threshold. If the event score meets the alert threshold, the system may determine whether the clinician's outlier score meets a trustworthiness threshold. In such implementations, at step 360, in response to determining that a clinician's outlier score meets the trustworthiness threshold but a new prescription event is scored as suspicious (meets the alert threshold), the system may push an alert to the clinician requesting confirmation that the clinician did indeed order the prescription. This would enable a clinician to interrupt an illicit prescription before it is filled. In some implementations, the confirmation notification can be sent to addresses (e.g., email addresses, mobile phone numbers, etc.) associated with the NPI of the clinician. In some implementations, the confirmation notification can be sent to addresses associated with an account of the clinician. In some implementations the confirmation notification may ask the clinician to reply if the prescription was not ordered by the clinician and/or should not be fulfilled. If the system receives a response to the confirmation notification that the prescription event is not confirmed (e.g., was not ordered by the clinician), the system can notify the pharmacy of the result of the response.

The event score and an outlier score can be used to push potential fraud notifications to a physician. For example, bad actors can steal a physician's professional identity, e.g., by signing up as the physician under a practice and/or e-prescribing application not used by the physician. The bad actor may then begin writing illicit prescriptions using the different e-prescribing application without the physician's knowledge. But because an NPI number is needed for e-prescriptions, and because an NPI is unique to a physician, the scoring server 110 can trace the illicit prescription events back to the physician, and these events will impact the physician's outlier score, i.e., changing the score to reflect outlier behavior. Thus, a change in score (toward outlier behavior) over some predetermined period of time can trigger an alert to the physician. In some implementations, if an outlier score meets an alert threshold the system may refer (report) the physician for evaluation by a human expert. This report may be considered an alert to an enforcement expert review system. In some implementations, if a physician's outlier score meets a trustworthiness threshold but a new prescription event is scored as suspicious, the system may push an alert to the physician requesting confirmation that the physician did indeed order the prescription. This would enable a physician to interrupt an illicit prescription before it is filled. Method 300 may be performed by a scoring server, such as scoring server 110 of FIG. 1.

In addition to the configurations described above, an apparatus can include one or more apparatuses in computer network communication with each other or other devices. In addition, a computer processor can refer to one or more computer processors in one or more apparatuses or any combinations of one or more computer processors and/or apparatuses. An aspect of an embodiment relates to causing and/or configuring one or more apparatuses and/or computer processors to execute the described operations. The results produced can be output to an output device, for example, displayed on the display. An apparatus or device refers to a physical machine that performs operations, for example, a computer (physical computing hardware or machinery) that implement or execute instructions, for example, execute instructions by way of software, which is code executed by computing hardware including a programmable chip (chipset, computer processor, electronic component), and/or implement instructions by way of computing hardware (e.g., in circuitry, electronic components in integrated circuits, etc.)—collectively referred to as hardware processor(s), to achieve the functions or operations being described. The functions of embodiments described can be implemented in any type of apparatus that can execute instructions or code.

More particularly, programming or configuring or causing an apparatus or device, for example, a computer, to execute the described functions of embodiments creates a new machine where in case of a computer a general purpose computer in effect becomes a special purpose computer once it is programmed or configured or caused to perform particular functions of the embodiments pursuant to instructions from program software. According to an aspect of an embodiment, configuring an apparatus, device, computer processor, refers to such apparatus, device or computer processor programmed or controlled by software to execute the described functions.

A program/software implementing the embodiments may be recorded on a computer-readable media, e.g., a non-transitory or persistent computer-readable medium. Examples of the non-transitory computer-readable media include a magnetic recording apparatus, an optical disk, a magneto-optical disk, and/or volatile and/or non-volatile semiconductor memory (for example, RAM, ROM, etc.). Examples of the magnetic recording apparatus include a hard disk device (HDD), a flexible disk (FD), and a magnetic tape (MT). Examples of the optical disk include a DVD (Digital Versatile Disc), DVD-ROM, DVD-RAM (DVD-Random Access Memory), BD (Blu-ray Disk), a CD-ROM (Compact Disc-Read Only Memory), and a CD-R (Recordable)/RW. The program/software implementing the embodiments may be transmitted over a transmission communication path, e.g., a wire and/or a wireless network implemented via hardware. An example of communication media via which the program/software may be sent includes, for example, a carrier-wave signal.

The many features and advantages of the embodiments are apparent from the detailed specification and, thus, it is intended by the appended claims to cover all such features and advantages of the embodiments that fall within the true spirit and scope thereof. Further, since numerous modifications and changes will readily occur to those skilled in the art, it is not desired to limit the inventive embodiments to the exact construction and operation illustrated and described, and accordingly all suitable modifications and equivalents may be resorted to, falling within the scope thereof.

Those skilled in the art will recognize that the present teachings are amenable to a variety of modifications and/or enhancements. For example, although the implementation of various components described above may be embodied in a hardware device, it can also be implemented as a software only solution—e.g., an installation on an existing server. In addition, the dynamic relation/event detector and its components as disclosed herein can be implemented as a firmware, firmware/software combination, firmware/hardware combination, or a hardware/firmware/software combination.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

In some aspects, the techniques described herein relate to a method including: obtaining a prescription history for a clinician; obtaining an outlier score for the clinician by providing the prescription history to an outlier detection model, the outlier score representing a probability that the prescription history represents an outlier; determining whether the outlier score for the clinician meets an alert threshold; and in response to determining that the outlier score meets the alert threshold, denying an access request from the clinician to an electronic prescription application.

In some aspects, the techniques described herein relate to a method, further including: using the outlier score to obtain an event score for a prescription event associated with the clinician and a pharmacy, the event score being based the outlier score and attributes of the clinician, the pharmacy, a medication, and the prescription event; determining whether the event score meets an alert threshold; and in response to determining that the event score meets the alert threshold, providing an alert to the pharmacy.

In some aspects, the techniques described herein relate to a method, where the attributes of the clinician, the pharmacy, the medication, and the prescription event are modeled in a graph database, where nodes in the graph database represent clinicians, pharmacies, medications, or prescription events, with edges connecting prescription events to respective clinicians, pharmacies, and medications.

In some aspects, the techniques described herein relate to a method, wherein prescription events in the prescription history are associated with an account and the outlier score includes an outlier score for each account represented in the prescription history, and wherein the electronic prescription application is associated with an account that has an outlier score that meets the alert threshold.

In some aspects, the techniques described herein relate to a method, wherein prescription events in the prescription history are associated with a practice and the outlier score includes an outlier score for each practice represented in the prescription history.

In some aspects, the techniques described herein relate to a method, wherein the prescription history represents three months since a first prescription event associated with the clinician.

In some aspects, the techniques described herein relate to a method, wherein reinforcement learning based on clinicians positively identified as having an outlier prescribing history is used to further train the outlier detection model.

In some aspects, the techniques described herein relate to a method including: maintaining a graph database, where each node in the graph database represents one of a clinician, a pharmacy, a medication, or a prescription event, with edges connecting prescription events to respective clinician, pharmacy, and medication nodes; generating an event score for a prescription event by providing a clinician, a pharmacy, and a medication to a machine-learning model, the machine-learning model using the graph database to provide the event score as output; determining whether the event score meets an alert threshold; and in response to determining that the event score meets the alert threshold, providing an alert to the pharmacy.

In some aspects, the techniques described herein relate to a method, wherein the graph database further stores nodes representing patients, a prescription event further including a link to a respective patient.

In some aspects, the techniques described herein relate to a method, wherein the graph database further stores nodes representing sponsors, a prescription event further including a link to a respective sponsor.

In some aspects, the techniques described herein relate to a method, wherein at least some of the nodes representing clinicians include an outlier score attribute for the clinician, the outlier score attribute representing a probability that a prescription history for the clinician represents an outlier.

In some aspects, the techniques described herein relate to a method, wherein the outlier score attribute is generated periodically by an outlier detection model.

In some aspects, the techniques described herein relate to a method, wherein the outlier score attribute is generated as part of generating the event score for the prescription event.

In some aspects, the techniques described herein relate to a method including: receiving a prescription event, the prescription event identifying a clinician, a pharmacy, and a medication; generating one or more vectors describing the prescription event, the clinician, the pharmacy, and the medication; generating an event score for the prescription event by providing the one or more vectors describing the prescription event, the clinician, the pharmacy, and the medication to a machine-learning model, the machine-learning model using the one or more vectors to provide the event score as output; determining whether the event score meets an alert threshold; and in response to determining that the event score meets the alert threshold, providing an alert to the pharmacy.

In some aspects, the techniques described herein relate to a method, wherein the one or more vectors include an outlier score for the clinician, the outlier score representing a probability that a prescription history for the clinician represents an outlier.

In some aspects, the techniques described herein relate to a method, further including, in response to determining that the event score meets the alert threshold: determining whether the outlier score meets a trustworthiness threshold; and in response to determining that the outlier score meets the trustworthiness threshold, providing a confirmation notification to the clinician.

In some aspects, the techniques described herein relate to a method, wherein the outlier score is generated periodically by an outlier detection model.

In some aspects, the techniques described herein relate to a method, wherein the outlier score is generated as part of generating the event score for the prescription event.

In some aspects, the techniques described herein relate to a method, wherein the prescription event further identifies a patient, and the one or more vectors further describes the patient, and wherein the one or more vectors describing the prescription event, the clinician, the pharmacy, the medication, and the patient are provided to the machine-learning model.

In some aspects, the techniques described herein relate to a method, wherein the prescription event further identifies a sponsor, and the one or more vectors further describes the sponsor, and wherein the one or more vectors describing the prescription event, the clinician, the pharmacy, the medication, the patient, and the sponsor are provided to the machine-learning model.

What is claimed is:

1. A method comprising:
obtaining a prescription history for a clinician;
obtaining an outlier score for the clinician by providing the prescription history to an outlier detection model, the outlier score representing a probability that the prescription history represents an outlier;
determining whether the outlier score for the clinician meets an alert threshold; and
in response to determining that the outlier score meets the alert threshold, denying an access request from the clinician to an electronic prescription application.

2. The method of claim 1, further comprising:
using the outlier score to obtain an event score for a prescription event associated with the clinician and a pharmacy, the event score being based the outlier score and attributes of the clinician, the pharmacy, a medication, and the prescription event;
determining whether the event score meets an alert threshold; and
in response to determining that the event score meets the alert threshold, providing an alert to the pharmacy.

3. The method of claim 2, where the attributes of the clinician, the pharmacy, the medication, and the prescription event are modeled in a graph database, where nodes in the graph database represent clinicians, pharmacies, medications, or prescription events, with edges connecting prescription events to respective clinicians, pharmacies, and medications.

4. The method of claim 1, wherein prescription events in the prescription history are associated with an account and the outlier score includes an outlier score for each account represented in the prescription history, and wherein the electronic prescription application is associated with an account that has an outlier score that meets the alert threshold.

5. The method of claim 1, wherein prescription events in the prescription history are associated with a practice and the outlier score includes an outlier score for each practice represented in the prescription history.

6. The method of claim 1, wherein the prescription history represents three months since a first prescription event associated with the clinician.

7. The method of claim 1, wherein reinforcement learning based on clinicians positively identified as having an outlier prescribing history is used to further train the outlier detection model.

8. A method comprising:
maintaining a graph database, where each node in the graph database represents one of a clinician, a pharmacy, a medication, or a prescription event, with edges connecting prescription events to respective clinician, pharmacy, and medication nodes;
generating an event score for a prescription event by providing a clinician, a pharmacy, and a medication to a machine-learning model, the machine-learning model using the graph database to provide the event score as output;
determining whether the event score meets an alert threshold; and
in response to determining that the event score meets the alert threshold, providing an alert to the pharmacy.

9. The method of claim 8, wherein the graph database further stores nodes representing patients, a prescription event further including a link to a respective patient.

10. The method of claim 8, wherein the graph database further stores nodes representing sponsors, a prescription event further including a link to a respective sponsor.

11. The method of claim 8, wherein at least some of the nodes representing clinicians include an outlier score attribute for the clinician, the outlier score attribute representing a probability that a prescription history for the clinician represents an outlier.

12. The method of claim 11, wherein the outlier score attribute is generated periodically by an outlier detection model.

13. The method of claim 11, wherein the outlier score attribute is generated as part of generating the event score for the prescription event.

14. A method comprising:
receiving a prescription event, the prescription event identifying a clinician, a pharmacy, and a medication;
generating one or more vectors describing the prescription event, the clinician, the pharmacy, and the medication;
generating an event score for the prescription event by providing the one or more vectors describing the prescription event, the clinician, the pharmacy, and the medication to a machine-learning model, the machine-learning model using the one or more vectors to provide the event score as output;
determining whether the event score meets an alert threshold; and
in response to determining that the event score meets the alert threshold, providing an alert to the pharmacy.

15. The method of claim 14, wherein the one or more vectors include an outlier score for the clinician, the outlier score representing a probability that a prescription history for the clinician represents an outlier.

16. The method of claim 15, further comprising, in response to determining that the event score meets the alert threshold:
determining whether the outlier score meets a trustworthiness threshold; and
in response to determining that the outlier score meets the trustworthiness threshold, providing a confirmation notification to the clinician.

17. The method of claim 15, wherein the outlier score is generated periodically by an outlier detection model.

18. The method of claim 15, wherein the outlier score is generated as part of generating the event score for the prescription event.

19. The method of claim 14, wherein the prescription event further identifies a patient, and the one or more vectors further describes the patient, and wherein the one or more vectors describing the prescription event, the clinician, the pharmacy, the medication, and the patient are provided to the machine-learning model.

20. The method of claim 19, wherein the prescription event further identifies a sponsor, and the one or more vectors further describes the sponsor, and wherein the one or more vectors describing the prescription event, the clinician, the pharmacy, the medication, the patient, and the sponsor are provided to the machine-learning model.
